(19) United States
(12) Patent Application Publication
(10) Pub. No.: US 2026/0326175 A1
(43) Pub. Date: Oct. 22, 2026

(54) METHOD FOR THE TREATMENT OF ORGANIC WASTE

(71) Applicant: SUEZ INTERNATIONAL, Paris La Défense Cedex (FR)

(72) Inventors: Pierre-Emmanuel PARDO, Paris La Défense Cedex (FR); Safwan AL AYOUBI, Paris La Défense Cedex (FR)

(73) Assignee: SUEZ INTERNATIONAL, Paris La Défense Cedex (FR)

(21) Appl. No.: 18/997,914

(22) PCT Filed: Jul. 28, 2023
(86) PCT No.: PCT/EP2023/071014

§ 371 Date: Jan. 23, 2025

(30) Foreign Application Priority Data
Jul. 29, 2022 (FR) FR2207895

(51) Int. Cl. B09B 3/65; B09B 3/40; B09B 101/70; B09B 101/85; C02F 11/04; C02F 11/18; C12P 5/02

(52) U.S. Cl. CPC B09B 3/65; B09B 3/40; C02F 11/04; C02F 11/18; C12P 5/023; B09B 2101/70; B09B 2101/85

(57) ABSTRACT

The invention relates to a treatment method comprising:
a step of feeding a first, easily fermentable stream M2 and a second, not easily fermentable stream M3
a step of hydrothermally gasifying at least one fraction of the second, not easily fermentable stream M3 in order to obtain a stream of mineral matter M4, a stream of organic matter M5 and gas
a digestion DG1 carried out on at least one fraction of the first easily fermentable stream M2, making it possible to obtain a stream M61 and gas, and
a digestion DG2 carried out on at least one fraction of the stream of organic matter M5 from the hydrothermal gasification step, making it possible to obtain a stream M62 and gas.

### TECHNICAL FIELD OF THE INVENTION

The invention relates to the field of complex waste treatment and recovery.

### STATE OF THE ART

Against the backdrop of climate change and increasing scarcity of resources, the search for treatment methods that make the best possible use of energy from the various wastes produced by human activity is in full swing.

These technologies are diverse, and can be divided into “low-tech” and “high-tech” products.

“Low-tech” products enable energy production and resource recovery at low cost, but with generally low yields.

“High-tech” products enable energy production and resource recovery with high yields, but often at very high cost.

State-of-the-art methods do not generally enable all of these products to be recovered in an industrially reliable way, in particular by keeping down production costs and energy expenditure.

The present invention proposes a joint use of these “low-tech” and “high-tech” products to achieve a synergy that will find a technical and economic optimum for making the most of biomass while controlling production costs.

“Low-tech” products include, for instance, anaerobic digestion of biomass. Digestion is a robust product, but for certain products (e.g. lignin), yields are so low that building equipment to produce very little gas will not generate any return on investment, and so will be of no industrial interest.

On the other hand, a “high-tech” product applied to a product that could have been treated with a good yield using a “low-tech” product will be of little economic interest compared to the “low-tech” product. The difference in yield between the two technologies in this case will not justify the difference in investment. In other words, the marginal difference in production will be too small for the marginal difference in investment. An example of a “high-tech” product is hydrothermal gasification, which can produce a high proportion of gas in relation to the input product, but at a very high cost.

By combining digestion and hydrothermal gasification in an intelligent (optimal) way, optimum gas production efficiency is achieved while minimizing investment costs.

### SUMMARY OF THE INVENTION

The invention relates to a treatment method comprising:
A step of feeding a first, easily fermentable stream M2 and a second, not easily fermentable stream M3
A step of hydrothermally gasifying at least one fraction of the second, not easily fermentable stream M3 in order to obtain a stream of mineral matter M4, a stream of organic matter M5 and gas,
A digestion DG1 carried out on at least one fraction of the first easily fermentable stream M2, making it possible to obtain a stream M61 and gas, and
A digestion DG2 carried out on at least one fraction of the stream of organic matter M5 from the hydrothermal gasification step, making it possible to obtain a stream M62 and gas,
it being understood that the digestion steps DG1 and DG2 can be carried out in two different digesters or in the same digester,
said first easily fermentable stream M2 is characterized in that mesophilic digestion at 35-40° C. for 20 days reduces the organic matter present in said first stream by more than 40% by weight, and
said second not easily fermentable stream M3 is characterized in that mesophilic digestion at 35-40° C. for 20 days does not enable more than 40% by weight of the organic matter present in said second stream to be converted into biogas.

In one embodiment of the method of the invention:
the digestions DG1 and DG2 are carried out in two different digesters, said digestion DG1 allowing to obtain a digestate M61 and gas, said digestion DG2 allowing to obtain a digestate M62 and gas, said method further comprising a step of mixing at least one fraction of the stream M62 and at least one fraction of the stream M61 in order to obtain a stream M63, or
the digestions DG1 and DG2 are carried out in the same digester, producing digestate M6 and gas.

In one embodiment, the method of the invention further comprises, upstream of the hydrothermal gasification step, a step of separating a mixture M1 comprising at least organic matter to obtain the first easily fermentable stream M2 on the one hand and the second not easily fermentable stream M3 on the other hand.

In one embodiment, the method of the invention further comprises, downstream of digestion, a dewatering step carried out on at least one fraction of the digestate M6 or on at least one fraction of the stream M63, in order to obtain a dewatered organic matter stream M7.

Preferably, all of the digestate M6 or the entire stream M63 is fed into a dewatering device for the dewatering step.

Preferably, at least one fraction of the dewatered organic matter stream M7 is recycled upstream of the hydrothermal gasification step.

In one embodiment of the method of the invention, the entire first easily fermentable stream M2 is used for digestion DG1.

In one embodiment of the method of the invention, the entire second not easily fermentable stream M3 is used in the hydrothermal gasification step.

In one embodiment of the method of the invention, the hydrothermal gasification step is carried out at a pressure ranging from 200 to 450 bar, more preferably from 250 to 300 bar and/or at a temperature ranging from 350 to 600° C., preferably from 400 to 550° C.

In one embodiment of the method of the invention, the hydrothermal gasification step comprises a separation step making it possible to obtain on the one hand a stream M4 enriched in inorganic matter and a stream M3′ depleted of inorganic matter, and a step of cooling said stream M3′ and a step of expansion of said cooled stream M3′ in order to obtain a gas and the stream M5, said cooling step being preferably implemented by heat exchange with the fraction of the second stream M3 which is not easily fermentable.

In one embodiment of the method of the invention, the digestions DG1 and DG2 are implemented in the same digester and the fraction of the organic matter stream M5 is introduced into a recirculation loop of said digester in which a stream M6′ circulates from a digester outlet to a digester inlet.

In one embodiment, the method of the invention further comprises, downstream of the hydrothermal gasification step and upstream of digestion, a step of cooling the fraction of the organic matter stream M5 to a temperature of less than or equal to 60° C., preferably less than or equal to 40° C., prior to its introduction into the digester for the digestion step.

In one embodiment of the method of the invention, at least one fraction of the organic matter stream M5 from the hydrothermal gasification step is introduced into a digester for digestion, without prior cooling.

In one embodiment of the method of the invention, the digestion is anaerobic digestion.

In one embodiment of the method, the fraction of the first easily fermentable stream M2 and the fraction of the organic matter stream M5 are mixed upstream of the digestion step to obtain a mixture M8 which is introduced into the digester, with digestions DG1 and DG2 being carried out in the same digester.

The inventors discovered that hydrothermal gasification does not always achieve 100% organic matter conversion yields, and that it is advantageous not to push the thermal reaction conditions of hydrothermal gasification to reduce costs, and to use anaerobic digestion to complete the conversion of organic matter into gas.

### BRIEF DESCRIPTION OF THE FIGURES

FIG. 1 shows an example of an installation for implementing the treatment method described in the invention.

FIG. 2 shows an example of an installation for implementing the treatment method described in the invention.

FIG. 3 shows an example of an installation for implementing the treatment method described in the invention.

FIG. 4 shows an example of an installation for implementing the treatment method described in the invention.

FIG. 5 shows an example of an installation for implementing the treatment method described in the invention.

FIG. 6 shows an example of an installation for implementing the treatment method described in the invention.

### DETAILED DESCRIPTION OF THE INVENTION

The invention relates to a treatment method comprising:
a step of feeding a first, easily fermentable stream M2 and a second, not easily fermentable stream M3
a step of hydrothermally gasifying at least one fraction of the second, not easily fermentable stream M3 in order to obtain a stream of mineral matter M4, a stream of organic matter M5 and gas
a digestion DG1 carried out on at least one fraction of the first easily fermentable stream M2, making it possible to obtain a stream M61 and gas, and
a digestion DG2 carried out on at least one fraction of the stream of organic matter M5 from the hydrothermal gasification step, making it possible to obtain a stream M62 and gas,
it being understood that the digestion steps DG1 and DG2 can be carried out in two different digesters or in the same digester.

In one embodiment of the treatment method:
the digestions DG1 and DG2 are carried out in two different digesters, said digestion DG1 allowing to obtain a digestate M61 and gas, said digestion DG2 allowing to obtain a digestate M62 and gas, said method further comprising a step of mixing at least one fraction of the stream M62 and at least one fraction of the stream M61 in order to obtain a stream M63, or
the digestions DG1 and DG2 are carried out in the same digester, producing digestate M6 and gas.

The invention thus relates to the treatment of a set of biomass of which a part of the biomass is easily fermentable and another part of the biomass is not easily fermentable.

For the purposes of the present invention, the expression “at least a fraction of a mixture or stream” has the same meaning as the expression “all or part of said mixture or stream”. When referring to a part of said mixture or stream, this expression refers to a certain proportion of said mixture or stream. For example, in the sense of this expression, “each fraction of the mixture” or “each fraction of the stream” will have the same composition.

Thus, for the purposes of the present invention, the expression “step X implemented on the entire stream M from step Y” means that steps X and Y are successive and that there is no intermediate separation step between steps X and Y, it being understood that the stream M could be cooled/heated between steps X and Y, in particular by heat exchange which could possibly involve a stream exchange.

### Stream M2 and Stream M3

For the purposes of the present invention, the first easily fermentable stream M2 is characterized in that mesophilic digestion at 35-40° C. for 20 days reduces the organic matter present in said first stream by more than 40% by weight.

For the purposes of the present invention, the second not easily fermentable stream M3 is characterized in that mesophilic digestion at 35-40° C. for 20 days does not enable more than 40% by weight of the organic matter present in said second stream to be converted into biogas.

These characterizations are based on best practices for measuring the methane potential of a substrate (Biochemical Methane Potential), known to those skilled in the art, which enable the gas production of a substrate to be measured under mesophilic conditions. These best practices are mentioned, for example, in the NF ISO 19388:2023 standard.

The first easily fermentable stream M2 can be chosen from biomasses from food waste collection, fats and edible oils, for example, or the fraction recovered by primary decantation from a water treatment plant. In particular, this can be the part of the organic matter resulting from primary decantation at the treatment plant inlet.

The second stream M3, which is not easily fermentable, can be selected from lignocellulosic waste (e.g. manure, straw, cellulosic agricultural waste) and biological sludge from wastewater treatment plants. This may particularly include biomass from a wastewater treatment plant after biological treatment.

For example, in the case of wastewater treatment plants, the so-called “primary treatment” steps, which consist of simple decantation of the input organic matter, generally produce an easily fermentable residue in a digester, with yields that can typically exceed 50% of organic matter transformation.

On the other hand, the “biological treatments” that follow the “primary treatments” will generally produce a residue with a digestion yield that can fall below 30%.

In one embodiment, the treatment method according to the invention comprises a step of separating a mixture M1 comprising at least organic matter to obtain the first easily fermentable stream M2 on the one hand and the second not easily fermentable stream M3 on the other hand.

The separation step can, for example, be implemented by washing and decanting a mixture M1, thus creating two separate streams: an easily fermentable stream M2 and a not easily fermentable stream M3.

### The mixture M1 can be selected from manure and food waste.

For example, manure containing both straw and animal feces is generally made up of a part that is not easily fermentable (the straw) and a part that is easily fermentable (the feces).

Separating these two streams, for example by washing and decanting (straw does not solubilize in water, unlike fecal matter), can create two separate streams: one that is easily fermentable, the other that is not easily fermentable.

In one embodiment, the easily fermentable stream M2 comprises from 5 to 50% by weight of solids, preferably from 15 to 25% by weight of solids, based on the total weight of the mixture M2.

### Hydrothermal Gasification

The treatment method according to the invention comprises at least one hydrothermal gasification (HG) step carried out on at least a fraction of the second, not easily fermentable stream M3.

Hydrothermal gasification (HG) is a thermal depolymerization method used to convert organic matter present in a humid environment into a mixture comprising only small molecules under high to moderate temperature and pressure.

During the HG process, the carbon and hydrogen in an organic matter are thermochemically converted under near-critical or supercritical conditions. One part is converted into water-soluble compounds with low molar masses.

Another part is converted into gaseous products such as carbon dioxide (CO2), methane (CH4), dihydrogen (H2), carbon monoxide (CO), light hydrocarbons such as ethane (C2H6) and propane (C3H8).

During residence in the hydrothermal gasification reactor at temperatures below 400° C., the organic matter undergoes, among other reactions, hydrolysis-based decomposition, similar to the reactions occurring in the liquefaction method, but much more rapidly. This is because implementation under near-critical or supercritical conditions makes it possible to use the unique properties of supercritical water as a solvent, enabling homogeneous solvation and reaction conditions, leading to very high reaction kinetic speeds. As a result, a much shorter residence time and a much higher heating rate than in conventional hydrolysis are used, limiting or even avoiding the secondary condensation and polymerization reactions responsible for bio-oil and bio-char formation.

When the HG process runs at temperatures above 400° C., radical decomposition of polymers (involving in particular decarboxylation, deamination by breaking C—N bonds, and C—C or C—O cleavage) predominates, while endothermic steam reforming is the main reaction pathway for converting small molecules with 1-3 carbon atoms into carbon oxides and dihydrogen, and nitrogen into ammonia.

Methane is also produced by the methanation of CO and CO2, using dihydrogen.

Consequently, HG can be seen as a decomposition method transforming the organic residue present in the stream M3 into a more readily biodegradable matter and ammonia dissolved in the liquid phase.

The treatment conditions (in particular temperature, pressure, and to a lesser extent residence time) of the HG can be adjusted to not only produce a gaseous fraction containing CH4, CO, CO2 and H2 (syngas), but also to produce an aqueous effluent, containing mainly easily digestible compounds, notably carboxylic acids, on the one hand, and ammonia in the form of the ammonium salt of the carbonic acids produced, on the other.

It should be noted that HG is different from hydrothermal liquefaction (HTL), in particular in that the conversion rate and level of decomposition of organic matter in HTL are not as high as in HG, even when HG is carried out under moderate temperature conditions.

Under HTL conditions, the water still contains HO− and H3O+ ions, which initiate the hydrolysis of organic matter.

Hydrolysis only takes place on the surface of the cellulose compounds contained in the organic fraction, which dissolves very little in the subcritical medium, resulting in fairly low decomposition conversions.

Condensation reactions (comprising mainly Aldol condensation, alkylation or Friedel-Craft acylation) of intermediates are an important reaction pathway, leading to the formation of biocrude which is an oil (also called bio-oil) that can be used as a fuel, i.e. biocrude contains organic molecules containing 5 or more carbon atoms, typically from 8 to 16 carbon atoms. On the other hand, the liquid product of HG, on the other hand, contains mainly readily biodegradable compounds.

HG differs from pyrolysis in that it is carried out in a medium containing water, the water being in a supercritical or near-critical state.

HG differs from “conventional” gasification of organic matters in that “conventional” gasification reduces the carbon/hydrogen (C/H) mass ratio, resulting in products with increased calorific value, including a gas composed mainly of synthesis gas (mixture of H2/CO), bio-oil and/or carbonaceous solid (char).

This HG step produces a stream of mineral matter M4, a stream of organic matter M5 and gas.

The mineral matter stream M4 will typically comprise a higher proportion of mineral matter than the proportion of mineral matter in the stream M3.

The organic matter stream M5 will typically comprise a higher mass ratio of mineral matter than the mass ratio of organic matter in the stream M3.

Also typically, the mineral matter stream M4 will comprise a higher proportion of mineral matter than the proportion of mineral matter in the stream M5.

Also typically, the organic matter stream M4 will comprise a higher proportion of organic matter than the proportion of organic matter in the stream M5.

Hydrothermal gasification is the term used to describe the entire processing chain leading, on the one hand, to the production of a gas through the transformation of organic matter and, on the other, to a plurality of liquid fractions containing more or less inorganic matter.

Typically, hydrothermal gasification involves at least pressurizing and tempering the biomass, in particular at temperatures above 350° C. and at pressures preventing vaporization of the medium.

In one embodiment, hydrothermal gasification is carried out in a gasification reactor:
at a temperature ranging from 350° C. to 600° C., preferably 350° C. to 550° C., and/or
at a pressure of 200 to 450 bar, preferably 250 to 300 bar.

Hydrothermal gasification may also comprise separation of the inorganic fraction (salts) and may involve the use of catalysts.

Inorganic matter comprises salts comprising anions such as phosphates, sulfates, chlorides, carbonates and hydrocarbonates, with counterions such as sodium, magnesium, calcium, ammonium and metals.

Preferably, the (overall) residence time of the inorganic matter-depleted stream M4 in HG step d) typically ranges from 1 min to 20 min, preferably from 2 min to 10 min, more preferably from 3 to 5 min.

In one embodiment, the hydrothermal gasification step is carried out in a gasification reactor in the presence of at least one catalyst. Preferably, the catalyst is chosen from metals on activated carbon, such as ruthenium, nickel, palladium or platinum.

The catalyst can be in the form of a bed of solid particles within the gasification reactor.

The hydrothermal gasification step will thus generally lead to a mixture comprising inorganic matter (salts) and organic matter. In the context of the invention, the HG step typically includes a separation step to obtain a stream M4 enriched in inorganic matter and a stream M3′ depleted of inorganic matter. The M3′ stream will then generally be a stream of gas dissolved in a liquid effluent.

For the purposes of the present invention, when separating a given stream X, “a stream enriched with inorganic matter” or “inorganic-enriched stream” means a stream comprising a mass ratio of inorganic matter greater than the mass ratio of inorganic matter in the given stream X, these mass ratios being defined as a proportion of dry matter.

For the purposes of the present invention, when separating a given stream X, the term “stream depleted of inorganic matter” or “inorganic-depleted stream” means a stream comprising a mass ratio of inorganic matter lower than the mass ratio of inorganic matter in the given stream X, these mass ratios being defined as a proportion of dry matter.

Separation of the M3′ and stream M4s can be achieved by separation, which can occur by gravitational precipitation during the hydrothermal gasification process of the mixture of the HG reactor.

Typically, the HG step comprises a step of cooling the stream M3′ before an expansion step to obtain a gas on the one hand and the liquid stream M5 on the other. In one embodiment, said cooling step is carried out by heat exchange with the fraction of the second, not easily fermentable stream M3 (upstream of the HG). In this way, the not easily fermentable stream M3 upstream of the HG step recovers the heat present in the stream M3′, thereby cooling the stream M3′ and heating the stream M3.

During the hydrothermal gasification step, a stream M4 enriched with inorganic matter is specifically recovered. Preferably, the ratio between the concentration of inorganic matter in M4 and the concentration of inorganic matter in M3 is at least 2, more preferably at least 5.

At the end of hydrothermal gasification, after any internal heat recovery, the outlet stream may have a high temperature (above 150° C.), and the gas and liquid are typically separated, resulting in a gas stream and a stream of organic matter M5. This separation can be carried out at atmospheric pressure or under pressure.

The hot liquid fraction M5 contains organic matter (organic matter stream) and can therefore be returned to the digester to heat same. The bacteria present in the digester may require heat (37-55° C.) for their metabolic activity.

This return of potentially high heat is best achieved under conditions that prevent thermal shock.

This mixing can take place, for example, in a digester sludge recirculation loop with a high flow rate plus a small flow rate of hot liquid fraction.

In one embodiment, the method further comprises, downstream of the hydrothermal gasification step and upstream of the digestion step, a step of cooling the fraction of the organic matter stream M5 to a temperature of less than or equal to 60° C., preferably less than or equal to 40° C., prior to its introduction into a digester for the digestion step.

### Digestion

The treatment method according to the invention comprises:
a digestion DG1 carried out on at least one fraction of the first easily fermentable stream M2, making it possible to obtain a stream M61 and gas, and
a digestion DG2 carried out on at least one fraction of the stream of organic matter M5 from the hydrothermal gasification step, making it possible to obtain a stream M62 and gas,
it being understood that the digestion steps DG1 and DG2 can be carried out in two different digesters or in the same digester.

In the treatment method described here, the digestions DG1 and DG2 can be carried out in the same digester or in two different digesters.

Thus, the method of the invention comprises a digestion step selected from:
i. a digestion step DG1 carried out in a first digester on at least a fraction of the first easily fermentable stream M2 to obtain a stream M61 and gas, and a digestion step DG2 carried out in a second digester on at least one fraction of the organic matter stream M5 from hydrothermal gasification, to obtain a stream M62 and gas, or
ii. a digestion step DG carried out in a digester on at least one fraction of the first easily fermentable stream M2 and on at least one fraction of the organic matter stream M5 from hydrothermal gasification, to obtain a stream M6 and gas.

In one preferred embodiment, the digestion step is chosen from among a digestion step DG carried out in a digester on at least one fraction of the first easily fermentable stream M2 and on at least one fraction of the organic matter stream M5 from hydrothermal gasification, to obtain a digestate M6 and gas.

Typically, digestion takes place in a digester, also known as a tank.

Advantageously, the digestion(s) implemented in the method according to the invention are anaerobic digestions.

Preferably, the digestion(s) are carried out in the absence of oxygen at a temperature ranging from 20 to 60° C.

When mesophilic digestion is used, the temperature in the digester ranges from 33° C. to 37° C., with a residence time of 14 to 22 days. When the digestion process only treats purely liquid effluent, the residence time can be considerably reduced. It should be noted that the stream M5 from the HG will be a liquid stream.

When thermophilic digestion is used, the temperature in the digester ranges from 55° C. to 60° C., with a residence time of 8 to 12 days. When the digestion process only treats purely liquid effluent, the residence time can be considerably reduced.

Residence time and temperature are two factors influencing the proper degradation of sludge, and therefore the optimization of energy production.

At the end of the digestion step, a biogas is obtained.

As used here, “anaerobic treatment” is understood to mean anaerobic digestion producing biogas. Anaerobic treatment is typically carried out in an anaerobic tank.

Anaerobic digestion can be mesophilic or thermophilic.

This biogas typically comprises a mixture essentially consisting of methane, carbon dioxide and water. The biogas may optionally comprise other gases, such as hydrogen, oxygen, nitrogen, hydrogen sulfide, but these other gases collectively represent less than 10% by weight of the biogas, of the total weight of the biogas.

Anaerobic digestion is a process involving micro-organisms that break down biodegradable matter in the absence of oxygen.

This process produces a digestate and a gaseous fraction comprising methane, and typically consisting essentially of methane and CO2, also known as biogas.

Advantageously, anaerobic digestion is a digestion of effluents containing only soluble components, in particular soluble carbon, i.e. no longer containing suspended solids.

Advantageously, the suspended solids have been solubilized in the hydrothermal gasification step.

In the case where the method implements two digestions DG1 and DG2 in two different digesters, then the digestion implemented on the stream M2 can be implemented under different conditions to the digestion implemented on the stream M5. For example, the digestion carried out on the stream M5 may be of the UASB (upflow anaerobic sludge blanket digestion) type, i.e. treating soluble carbon.

For example, if the stream M2 contains a high proportion of solids (typically over 3% by weight of solids), the digestion carried out on the stream M2 will advantageously be of the perfectly mixed type, with a digester stirring system ensuring homogeneity within the system.

Anaerobic treatment is generally carried out under pH conditions of between 7.0 and 7.5, preferably between 7.0 and 7.2.

In an advantageous embodiment, the digestions DG1 and DG2 are carried out in the same digester, and the organic matter stream M5 from gasification is introduced into a recirculation loop in which a stream M6′ circulates from a digester outlet to a digester inlet. This is particularly advantageous when there is no cooling device between the hydrothermal gasification reactor and the digester.

Preferably, the flow rate D1 of the organic matter stream M5 into said digester recirculation loop M6′ is lower than the flow rate D2 of the digester sludge circulation in the circulation loop. For example, the D2/D1 ratio can be at least 3.

Typically, the flow rate of the stream M5 after hydrothermal gasification is not controlled.

The flow rate of the stream M6′ can be controlled and adjusted based on the flow rate of the stream M5.

In one embodiment, the method further comprises, downstream of the hydrothermal gasification step and upstream of the digestion, a step of cooling the fraction of the organic matter stream M5 to a temperature of less than or equal to 60° C., preferably less than or equal to 40° C., prior to its introduction into a digester for digestion. This additional cooling step can be carried out using industrial water, for example.

This additional cooling step on the stream M5 is preferably implemented in the event that the heat stream provided by M5 would lead to too high a temperature in the digester as a whole (in the overall mass of the digester). In this case, it will be advantageous to remove heat from the stream M5 before introducing it into the digester.

According to one method, the fraction of the first easily fermentable stream M2 and the fraction of the organic matter stream M5 from the HG are mixed upstream of the digestion step, to be introduced as a mixture M8 into the digester.

The additional cooling step for the stream M5 is preferably implemented when the stream M5 is mixed with the stream M2 upstream of the digester, and in particular when this M8 mixture would (in the absence of such cooling) lead to a temperature that is too high, typically above 45° C.

After digestion, the treatment method produces:
in the case of digestions DG1 and DG2 in two different digesters, a first digestate M61 (and gas) and a second digestate M62 (and gas), or
in the case of digestions DG1 and DG2 in the same digester (referred to as a digestion DG), digestate M6 (and gas).

Preferably, in the case of digestions DG1 and DG2 in two different digesters, the method further comprises a step of mixing at least one fraction of the M62 stream and at least one fraction of the M61 stream to obtain an M63 stream.

In one embodiment, in the case of DG1 and DG2 digestions in two different digesters, the method further comprises a step of mixing the gas streams from each digester, prior to a possible gas treatment step. In fact, if the method comprises a subsequent downstream gas treatment step, it would be advantageous to pool the treatments.

In one embodiment, in the case of DG1 and DG2 digestions in the same digester, the method may optionally comprise a step for treating the gases from said digester.

In one embodiment, the treatment method further comprises, downstream of digestion, a dewatering step carried out:
on at least a fraction of the digestate M6 from digestion DG, preferably on all of the digestate M6 from the digestion DG, or
on at least a fraction of the stream M63, preferably the entire stream M63.

Preferably, the dewatered matter from the dewatering process is fed into the gasification reactor or into the stream M3 upstream of the gasification reactor.

In one embodiment, the treatment method according to the invention comprises a step of recirculating the sludge (stream M6′) from the digester outlet to the digester inlet, said step being preferably implemented in a recirculation loop. In this case, the stream M5 is preferably introduced into the digester sludge recirculation loop.

Preferably, according to this embodiment, the treatment method comprises a step for controlling the flow rate of the sludge thus recirculated, and for adapting said flow rate based on the flow rate of the stream M5, the flow rate of the stream M5 then typically not being controlled.

A recirculation loop can be provided from the digester outlet to its inlet, enabling sludge to be recirculated from the digester outlet to its inlet. In this way, it is possible to introduce the stream M5 into the recirculation loop, which is then introduced into the digester via this loop, mixed with the sludge from the recirculation loop. In this embodiment, it becomes possible to provide a control unit to control the flow rate of the sludge in the recirculation loop and adapt it to the flow rate of the uncontrolled stream M5.

Injecting the stream M5 into the recirculated sludge, rather than directly into the digester, avoids thermal shock and excessive temperature rise in the digester. This flow rate control and adjustment further controls and limits the temperature rise in the digester.

Preferably, the flow rate of the recirculated sludge is adapted so as not to generate a temperature increase of more than 5° C. in the mixed stream, after introduction of the stream M5.

The treatment method according to the invention can be implemented in an installation.

The present invention further relates to an installation as such for implementing the treatment method.

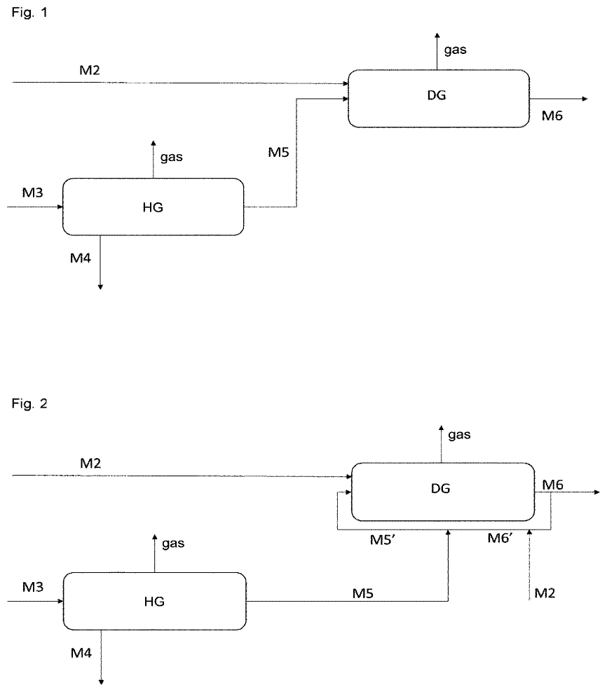

FIGS. 1 to 4 illustrate embodiments of an installation for carrying out the treatment method described herein, without limiting the scope thereof.

In one embodiment, the installation comprises:
A hydrothermal gasification reactor HG,
a digester DG with at least one digestate M6 outlet and one gas outlet,
A stream supply line connecting an outlet of the hydrothermal gasification reactor (stream M5) and an inlet:
   i. Either of a mixing device upstream of the digester,
   ii. Or of the DG digester,
A supply line that feeds:
   i. Or the mixing device, if present,
   ii. Or the digester DG,
possibly a dewatering device DH fed by the digestate line M6, comprising a water outlet and a dewatered stream M7 outlet,
optionally, a recycling loop for recycling at least a fraction of the dewatered M7 stream to the HG reactor inlet.

In another embodiment, the installation comprises:
A hydrothermal gasification reactor HG,
a digester DG1 with at least one digestate M61 outlet and one gas outlet,
A supply line of the stream M2 that feeds DG1,
a digester DG2 comprising at least one digestate M62 outlet and one gas outlet,
a stream supply line M5 connecting an outlet of the hydrothermal gasification reactor HG and an inlet of the digester DG2,
optionally a stream M63 line fed by digestate M61 line and digestate M62 line,
possibly a dewatering device DH supplied by the flow M63 line, comprising a water outlet and a dewatered stream M7 outlet,
optionally, a recycling loop for recycling at least a fraction of the dewatered M7 stream to the HG reactor inlet.

According to the installation shown in FIG. 1:
A supply line for the easily fermentable stream M2 feeds a digester DG,
A supply line for the not easily fermentable M3 stream feeds a hydrothermal gasification reactor HG, which has a stream M4 outlet for inorganic matter, a stream M5 outlet for organic matter and a gas outlet,
The stream outlet line M5 feeds the digester DG, which has a gas outlet and a digestate outlet M6.

In one embodiment shown in FIG. 2, the digester DG comprises a sludge recirculation loop, said sludge M6′ in the loop preferably having a sufficiently high flow rate to avoid a significant rise in the temperature of said loop, after introduction of the stream M5. For example, the temperature rise will preferably be less than or equal to 5° C. Thus, the flow rate of the stream M6′ in the loop will be adapted to the temperature and flow rate of the stream M5 to avoid this temperature rise in the stream M5′ (stream in the loop, after introduction of the stream M5).

In this case, the recirculation loop advantageously comprises a variable-speed pump for controlling the loop return temperature.

As shown in FIG. 2, the stream M2 can be introduced directly into the digester DG and/or into the recirculation loop, preferably upstream of M5 injection.

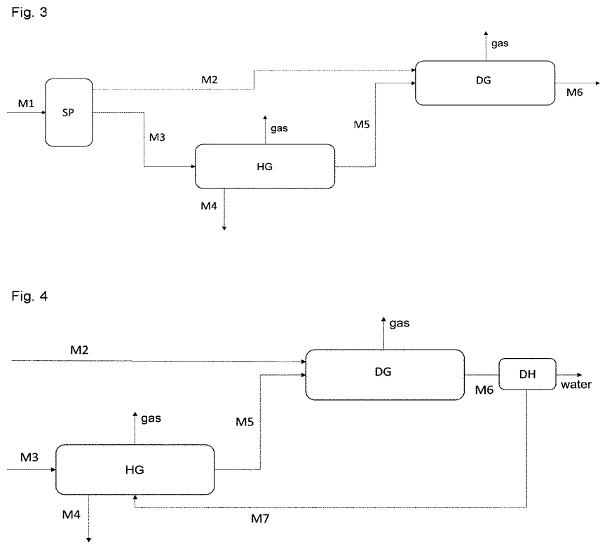

The embodiment shown in FIG. 3 differs from that of FIG. 1 in that the method comprises a preliminary step of separating a mixture M1. Thus, the installation according to this embodiment comprises a separation device SP fed by a stream supply line M1 and having two outlets, a stream outlet M2 and a stream outlet M3.

This method, which includes a preliminary separation step, can also be implemented as part of the method shown in FIG. 2, with a digester sludge recirculation loop.

The embodiment shown in FIG. 4 differs from that shown in FIG. 1 in that, downstream of the digestion step, the method also includes a digestate dewatering step prior to recycling into the gasification reactor. In this embodiment, the installation comprises a dewatering device DH fed by the stream M6 line and comprising a water outlet and a dewatered organic matter M7 outlet connected to an inlet of the gasification reactor.

This method, which includes a dewatering step, can also be implemented as part of the method shown in FIG. 2, with a digester sludge recirculation loop, and/or as part of the method shown in FIG. 3, with a preliminary separation step.

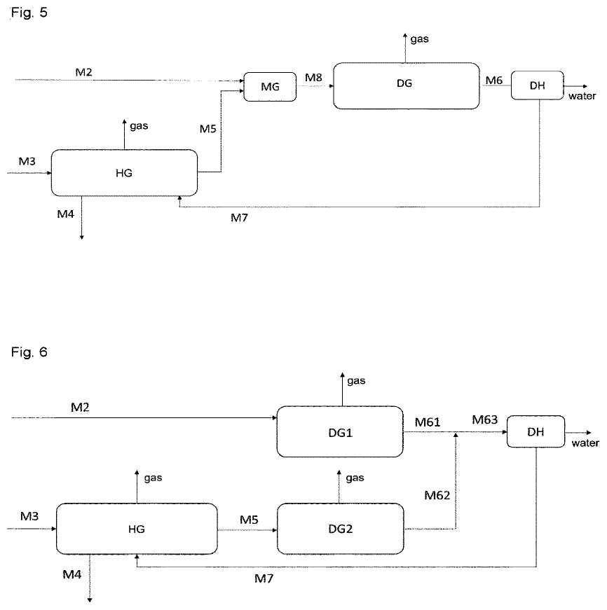

In one embodiment shown in FIG. 5, the installation includes a mixing device MG for mixing the stream M2 and the stream M5 from the HG. The mixed stream M8 from the mixing device is then introduced into the digester DG.

According to an embodiment not depicted, the installation comprises a cooling device downstream of the hydrothermal gasification reactor, fed by the stream M5 containing organic matter and enabling the M5 organic matter stream to be cooled to a temperature of less than or equal to 60° C., preferably less than or equal to 40° C., before being introduced into the digester for digestion.

In one embodiment not shown in the Figures, the gasification reactor HG will comprise
a separation device for obtaining the stream M4 and a stream M3′ of gas dissolved in a liquid effluent, and
a cooling device, preferably a heat recovery device, for cooling the stream M3′, and preferably for at least partially heating the stream M3 upstream of the reactor HG, and
an expansion device supplied with at least a fraction of the M3′ stream to obtain the gas stream on the one hand and the stream M5 (liquid effluent type) on the other.

In one embodiment shown in FIG. 6, the installation according to the invention comprises a DG1 digester fed by a stream M2 and a DG2 digester fed by at least a fraction of the stream M5 downstream of the HG reactor.

In this embodiment, the installation will advantageously comprise a device for mixing digestate M61 from DG1 and digestate M62 from DG2 to lead to a stream supply line M63.

According to this embodiment, the installation may also comprise, downstream of said mixing device, a dewatering device DH fed by the stream line M63 and comprising a water outlet and a dewatered organic matter outlet M7 connected to an inlet of the reactor HG.

In an unrepresented embodiment, the gas streams from DG1 and DG2 can be combined before being fed into a gas treatment unit.

The system according to the invention may of course comprise one or more of the features described as part of the method according to the invention.

1. A processing method, comprising:
feeding a first, easily fermentable stream M2 and a second, not easily fermentable stream M3,
hydrothermally gasifying at least one fraction of the second, not easily fermentable stream M3 to obtain a stream of mineral matter M4, a stream of organic matter M5, and gas,
carrying out a digestion DG1 on at least one fraction of the first easily fermentable stream M2, to obtain a stream M61 and gas, and
carrying out a digestion DG2 on at least one fraction of the stream of organic matter M5 from the hydrothermal gasification to obtain a stream M62 and gas,
wherein the digestion steps DG1 and DG2 are carried out in two different digesters or in the same digester,
wherein mesophilic digestion of the first easily fermentable stream M2 at 35-40° C. for 20 days reduces the organic matter present in the first stream by more than 40% by weight, and
wherein mesophilic digestion of the second not easily fermentable stream M3 at 35-40° C. for 20 days does not enable more than 40% by weight of the organic matter present in the second stream to be converted into biogas.

2. The treatment method according to claim 1, wherein:
the digestions DG1 and DG2 are carried out in two different digesters, the digestion DG1 producing a digestate M61 and gas, the digestion DG2 producing a digestate M62 and gas, and the method further comprises mixing at least one fraction of the stream M62 and at least one fraction of the stream M61 to obtain a stream M63, or
the digestions DG1 and DG2 are carried out in the same digester, producing digestate M6 and gas.

3. The method according to claim 1, further comprising, upstream of the hydrothermal gasification step:
separating a mixture M1 comprising at least organic matter to obtain the first easily fermentable stream M2 and the second not easily fermentable stream M3.

4. method according to claim 2, further comprising, downstream of digestion:
dewatering at least one fraction of the digestate M6 or on at least one fraction of the stream M63, to obtain a dewatered organic matter stream M7.

5. The method according to claim 4, wherein all of the digestate M6 or the entire stream M63 is introduced into a dewatering device for the dewatering.

6. The method according to claim 4, wherein at least a fraction of the dewatered organic matter stream M7 is recycled upstream of the hydrothermal gasification step.

7. The method according to claim 1, wherein the entire first easily fermentable stream M2 is used for digestion DG1.

8. The method according to claim 1, wherein all of the second stream M3, is used in the hydrothermal gasification.

9. The method according to claim 1, wherein the hydrothermal gasification is carried out at a pressure ranging from 200 to 450 bar and/or at a temperature ranging from 350 to 600° C.

10. The method according to claim 1, wherein the digestions DG1 and DG2 are implemented in the same digester, and the fraction of the organic matter stream M5 is introduced into a recirculation loop of said digester in which a stream M6′ circulates from a digester outlet to a digester inlet.

11. The method according to claim 1, further comprising:
cooling the fraction of the organic matter stream M5 to a temperature of less than or equal to 60° C. prior to its introduction into a digester for digestion, the cooling being downstream of the hydrothermal gasification step and upstream of digestion.

12. The method according to claim 1, wherein the hydrothermal gasification comprises:
separating to obtain a stream M4 enriched in inorganic matter and a stream M3′ depleted of inorganic matter;
cooling the stream M3′ to obtain a cooled stream M3′; and
expanding the cooled stream M3′ to obtain a gas and the stream.

13. The method according to claim 1, wherein at least one fraction of the organic matter stream M5 from the hydrothermal gasification is introduced into a digester for digestion, without prior cooling.

14. The method according to claim 1, wherein the digestion is anaerobic digestion.

15. The method according to claim 1, wherein the fraction of the first easily fermentable stream M2 and the fraction of the organic matter stream M5 are mixed upstream of the digestion to obtain a mixture M8 which is introduced into the digester, with digestions DG1 and DG2 being carried out in the same digester.

16. The method according to claim 9, wherein the hydrothermal gasification is carried out at a pressure ranging from 250 to 300 bar and/or at a temperature ranging from 400 to 550° C.

17. The method according to claim 12, wherein the cooling is implemented by heat exchange with the fraction of the second stream M3 which is not easily fermentable.
